(19) United States
(12) Patent Application Publication
(10) Pub. No.: US 2009/0022757 A1
(43) Pub. Date: Jan. 22, 2009

(54) Method for treating photoreceptor cell degeneration

(76) Inventors: Chee-Keung Chung, Kowloon (HK); Xin-Guo Deng, Kowloon (HK)

Correspondence Address: ANDREWS KURTH LLP;Intellectual Property Department, Suite 1100, 1350 I Street, N.W., Washington, DC, 20005, US

(21) Appl. No.: 11/826,952

(22) Filed: Jul. 19, 2007

(51) Int. Cl. A61K 36/06; A61P 27/02; A61P 43/00

(52) U.S. Cl. 424/195.15

(57) ABSTRACT

The present invention provides a method for treating photoreceptor cell degeneration in a mammal. The method includes orally administering a composition comprising germination activated sporoderm-broken Ganoderma lucidum spore oil (“GLS oil”) or a mixture of GLS oil and docosahexaenoic acid (DHA), to a mammal suffering from photoreceptor cell degeneration.

### FIELD OF THE INVENTION

This invention generally relates to a method for treating photoreceptor cell degeneration in retina. Specifically, the method includes orally administering oil extracted from germination activated sporoderm-broken Ganoderma lucidum spores (“GLS oil”) to a mammal suffering from photoreceptor cell damage in retina.

### BACKGROUND OF THE INVENTION

Photoreceptor cells are a specialized type of neuronal cells found in the eye's retina. They are responsible for transducing, or converting, light into nerve signals that can be ultimately transmitted to the brain via the optic nerve. The photoreceptor cells send signals to other neurons by a change in its membrane potential when it absorbs photons. The signals will be used by the visual system to form a complete representation of the visual world.

In vertebrates, there are two types of photoreceptor cells: rods and cones. Cones are adapted to detect colors, and function well in bright light; rods are more sensitive and adapted for low light, but do not detect color well. The human retina contains about 125 million rod cells and 6 million cone cells.

Retinitis pigmentosa (RP) is a human disease characterized by loss of photoreceptor cells, especially rods, leading to visual disturbance and eventually to blindness. It is the most common inherited cause of blindness in the developed world with an incidence of about 1/3500, and affecting about one million people all over the world (Kallonaitis M and Fletcher E L. Clin. Exp Optom, 2004, 87:65-80).

RP is a progressive, inherited disease inherited in an autosomal dominant, autosomal recessive, or X-linked fashion. It is characterized by a progressive loss of rod and cone photoreceptors leading to night blindness and can be diagnosed by changes in the electroretinogram (ERG). A gradual loss of visual acuity and peripheral vision leading to “tunnel” vision accompanies these changes, followed by loss of the central visual field in most cases. Fundus examinations in patients with RP reveal retinas with bone spicule formation resulting from melanin pigment that has infiltrated the retina (Boughman, J A, et al. 1980, Am J Hum Genet 32,223-235; Flannery, J G, et al. 1989, Invest Ophthalmol Vis Sci 30,191-211; van Soest, S, et al. 1999, Surv Ophthalmol 43,321-334). The first symptoms of RP usually appear in adolescence. Because the disease progresses slowly, individuals may not lose their central vision until the sixth decade of life (Berson, E L, 1996, Proc Natl Acad Sci USA 93,4526-4528). Mutations in more than 55 genes have been implicated in causing RP and associated syndromes (Daiger, S P et al. 2002, RetNet: Retinal Information Network at http//:www.sph.uth.tmc.edu/Retnet/home.htm). In most cases, regardless of the underlying mutation, the outcome of the disease is the same—photoreceptor cell death by apoptosis.

To date, there is still no cure or effective therapy for the treatment of RP. Experimental and clinical methods for RP treatment mainly includes gene therapy, drug therapy, transplant therapy and artificial retina implantation (Delyfer M N et al. Biology of the Cell, 2004, 96:261-269; Borras T, Exp Eye Res. 2003, 76:643-52). Accordingly, there exists an urgent need to develop methods and drugs for protecting retina and suppressing retinal degeneration.

Ganoderma belongs to the Polyporaceae group of the Fungi family. Ganoderma is widely used in traditional Chinese medicine as an auxiliary treatment for a variety of medical conditions, such as hepatis, AIDS, cancer, and autoimmune diseases. The germination activated sporoderm-broken Ganoderma lucidum spores (“GLSs”) are the essence of Ganoderma and contain active ingredients such as polysaccharides, sterols, oleic acid, linoleic acid, triterpenes, ceramides and certain organic ions, all of which possessing strong bioactivity. The GLSs have also been used for the treatment of neurological disorders.

GLS oil is prepared by a CO₂ supercritical extraction technology, that is, the extract is isolated in the CO₂ supercritical state of transformation from gas phase to liquid phase. Ganoderm lucidum spores oil contains all effective ingredients of triterpenes, polysaccharides and nucleotides. Ganoderm lucidum spores oil has broad pharmacological actions of immunoloregulation function, decreasing chemical and immune liver damage, anti oxidation and eliminating free radical and anti tumor (Liang J, et al. China Tropical Medicine. 2005, 5:1189-1191; Zhu W W, et al. Journal of First Military Medical University 2005, 25: 667-671; Tian G F, et al. China Oils and Fats, 2003, 28: 44-45).

Docosahexaenoic acid (DHA) is a ω-3 long chain unsaturated fatty acid in the biomembrane system. DHA is particularly rich in retina rod cell, cone cell and outer segment disc membrane, which plays important roles in the retinal development and function. The precursor of DHA is α-octadecatrienoic acid (linolenic acid), which is a fatty acid that cannot be synthesized in vivo and needs to be absorbed from food. The higher degree of unsaturation of DHA directly affects the biomembrane fluidity, further affecting some protein activity, biosignal transmission and receptor function. DHA is essential for the dynamic change of visual conductive cascade related proteins in the transmembrane process. In addition, DHA can change the biomembrane curvature, affect the membrane compression, and disrupt the hydrogen bond (Hu S X and Yang J N, Chinese Journal of Ophthalmology. 2003, 39: 251-253). Some studies found that DHA levels decreased in RP animal models of RP and RP patients.

It was reported that DHA had protective effects against kainic acid-induced retinal degeneration in rats (Mizota et al., Invest Ophthalmol Vis Sci., 2001, 42:216-221). Moriguchi et al. (Moriguchi et al., Ophthalmic Res. 2004, 36:98-105) also reported that DHA suppressed N-methyl-N-nitrosourea (MNU)-induced photoreceptor apoptosis in rats.

### SUMMARY OF THE INVENTION

The present invention provides a method for ameliorating photoreceptor cell degeneration in a mammal. The method comprises administering to a mammal suffering from photoreceptor cell degeneration an effective amount of germination activated sporoderm-broken Ganoderma lucidum spores oil (GLS oil). The photoreceptor cell degeneration can be caused by genetic factors, such as in retinitis pigmentosa (RP), by over-exposure to light, or by certain chemicals such as Kainate and MNU.

The effective amount of GLS oil to be used for ameliorating photoreceptor cell degeneration is between 0.2 and 20 mg/kg body weight/day.

Additionally, the GLS oil may be co-administered with an effective amount of DHA. The preferred amount of DHA to be used in conjunction with GLS oil is between 0.2 and 20 mg/kg body weight/day.

The GLS oil alone, or in combination with DHA, can ameliorate damages to photoreceptor cells in retina.

The present invention also provides a method for treating RP in a mammal. The method comprises administering to the mammal an effective amount of GLS oil. The GLS oil may be co-administered with an effective amount of DHA.

### BRIEF DESCRIPTION OF DRAWINGS

The patent application file contains at least one drawing executed in color. Copies of this patent application publication with color drawing(s) will be provided by the office upon request and payment of the necessary fee.

FIG. 1 is a composite of electroretinograms from rats treated with different doses of N-methyl-N-nitrosourea (MNU). Panels A, B, C, D, F are electroretinograms of rats treated with 30 mg/kg, 35 mg/kg, 40 mg/kg, 45 mg/kg and 50 mg/kg of MNU, respectively, at day 3. Panels A1, B1, C1, D1, F1 are electroretinograms of rat models treated with 30 mg/kg, 35 mg/kg, 40 mg/kg, 45 mg/kg and 50 mg/kg of MNU, respectively, at day 10. Panel G is the electroretinogram of a normal rat.

FIG. 2 is a composite of eye biopsy from rats treated with different doses of MNU. Panels A, B, C, D, E are biopsy of eyes from rats treated with 30 mg/kg, 35 mg/kg, 40 mg/kg, 45 mg/kg and 50 mg/kg of MNU, respectively, at day 3. Panels F and G are biopsy of eyes from rats treated with 35 mg/kg and 50 mg/kg of MNU, respectively, at day 10. Panel I is an eye biopsy from the normal control rat. INL: Inner Nuclear Layer; ONL: Outer Nuclear Layer.

FIG. 3 is a composite of electroretinograms from rats treated with different drugs. The rats had MNU-induced retinal photoreceptor cell degeneration. Panels A, B, C, and D are electroretinograms from rats in the GLS oil treatment group, DHA treatment group, GLS oil/DHA treatment group, and model control group, respectively, at day 3. Panels A1, B1, C1, D1 are electroretinograms from rats in the GLS oil treatment group, DHA treatment group, GLS oil/DHA treatment group, and model control group, at day 10, respectively. Panel E is the electroretinograms of a normal rat.

FIG. 4 is a composite of eye biopsy from rats treated with different drugs. The rats had MNU-induced retinal photoreceptor cell degeneration. Panels A, B, C, D are biopsy of eyes from rats in the GLS oil treatment group, DHA treatment group, GLS oiVIDHA treatment group, and model control group, respectively, at day 3. Panels A1, B1, C1, D1 are biopsy of eyes from rats in the GLS oil treatment group, DHA treatment group, GLS oil/DHA treatment group, and model control group, respectively, at day 7. INL: Inner Nuclear Layer; ONL: Outer Nuclear Layer.

### DETAILED DESCRIPTION OF THE INVENTION

There are three classes of animal models for retinal degeneration: animal models of inherited retinal degeneration, animal models with light-induced retinal photoreceptor cell degeneration, and animal models with N-methyl-N-nitrosourea (MNU)-induced retinal photoreceptor cell degeneration (Chader G J, Vision Research, 2002, 43:393-396; Deng X G, et al. Chinese Ophthalmic Research. 2006, 24 (1): 30-32; Yu T and Yin Z Q. Chinese Journal of Comparative Medicine. 2005, 15:120-123; Yu T and Yin Z Q, China Compative Med. J. 2005, 15:120-123; Wenzel A et al. Prog Retin Eye Res. 2005, 24:275-306; Yoshizawa K and Tsubura A., Nippon Ganka Gakkai Zasshi. 2005, 109: 327-37). The retina degeneration in all these animal models are caused by the apoptosis of the photoreceptor cell. Calcium over loading, free radical and oxidation damage also play crucial roles. It has been reported that calcium antagonists, neurotrophic factors, and anti apoptosis treatment could delay the apoptosis of the retinal photoreceptor cells. It has also been reported that anti-oxidative reagents could inhibit light-induced apoptosis of retinal photoreceptor cells, and that niacinamide and docosahexaenoic acid (DHA) have a therapeutic effect on MNU-induced retinal photoreceptor cell degeneration (Deng X G, et al., Chinese Traditional and Herbal Drugs. 2006, 37: 236-238; Takano Y et al. Biochem Biophys Res Commun, 2004. 313:1015-22; Kiuchi K et al. Curr Eye Res. 2003, 26:355-362; Moriguchi K, Y et al. Ophthalmic Res. 2004, 26: 98-105).

MNU is a nitroso compound distributed widely in the environment. MNU has strong carcinogenic, teratogenic and mutagenic activities, and has been used to induce breast carcinoma and reproductive organ tumors in animal models. In 1967, Herrold et al. first found that MNU could cause rat retinal degeneration. A single intraperitoneal injection of MNU would result in retinal photoreceptor cell damage and death due to photoreceptor cell apoptosis, downward adjustment of Bcl-2, and upward adjustment of Bax and activation of Caspase pathway. The MNU model is an ideal animal model for retinal degeneration research (Yoshizawa K et al. Lab Invest. 1999, 79: 1359-1367). Since retinal cell degenerations in experimental animals and humans are all caused by apoptosis of photoreceptor cell apoptosis, suppression of MNU-induced photoreceptor cells apoptosis in animals may provide therapeutic information for controlling retinal degeneration, such as RP, in humans.

As will be shown in the following examples, infra, MNU-related pathological changes in photoreceptor cells can be ameliorated by oral administration of GLS oil or a combination of GLS oil and DHA.

In describing preferred embodiments of the present invention, specific terminology is employed for the sake of clarity. However, the invention is not intended to be limited to the specific terminology so selected. It is to be understood that each specific element includes all technical equivalents which operate in a similar manner to accomplish a similar purpose.

One aspect of the present invention relates to a method for preventing or treating photoreceptor cell degeneration in a mammal. The method comprises administering to a mammal an effective amount of GLS oil.

GLS is a brown powder that is slightly soluble in water. Bioactive GLS can be produced in a process containing the following steps:

I. Induction of germination: Mature and perfect spores of red Ganoderma lucidum are carefully selected to undergo a soaking process to induce germination. Spores are kept in clear or distilled water, biological saline solution, or other nutritional solutions that could enable the spores of Ganoderma lucidum to germinate rapidly. Examples of nutritional solutions include coconut juice or a 1-5% malt extract solution, 0.5-25% extracts of Ganodenna lucidum sporocarps or Ganoderma lucidum capillitia, a solution containing 0.1-5% biotin, and a solution containing 0.1-3% potassium phosphate (monobasic) and magnesium sulfate. The choice of solution would depend on the soaking time required, the amount of spores to be processed and other such factors as availability of materials. One or more of the above germination solutions could be used, with the amount added being 0.1-5 times the weight of the spores of Ganoderma lucidum. The soaking time can be determined according to the temperature of the water, and usually the soaking was carried out for 30 min to 8 hours with the temperature of the water at 20-43° C. Preferably, the soaking time is 2-4 hours, and the temperature of water is 25-35° C.

II. Activation culture: The spores of Ganoderma lucidum are removed from the soaking solution and excess solution is eliminated by allowing it to drip. The spores are then placed in a well-ventilated culturing box at a constant temperature and humidity so that spore culture activation could be carried out. The relative humidity of the culture is generally set at 65-98%, the culture temperature set at 18-48° C. and the activation time may last from 30 min to 24 hours. Preferably humidity is 85-97% and temperature is 25-35° C. During activation, the cell walls of the spores of Ganoderma lucidum are clearly softened such that it is easier to penetrate the cell walls of the spores. The activation of spores of Ganoderma lucidum typically reaches a rate of more than 95%.

III. Treatment of the epispores: After the germination/activation process, the spores are treated by enzymolysis. This process is carried out at a low temperature and under conditions such that enzyme activity is maintained, using chitinase, cellulase, or other enzymes, which are commonly used in the industry. The process is complete when the epispores lost their resilience and became brittle. Alternatively, physical treatments are carried out to penetrate the cell walls, for example, micronization, roll pressing, grinding, super high pressure microstream treatment, and other mechanical methods commonly used in the industry could be carried out, with a penetration rate of over 99%.

IV. Drying or extraction: Drying is carried out at low temperature using standard methods including freeze-drying or vacuum-drying etc., which are commonly used in the industry. The obtained product has a moisture content less than 4%. After drying, the bioactive substances are extracted by water or alcohol, or by thin film condensation. The extracted bioactive substances can be further purified by dialysis to ensure no contamination in the final products. The final product can be made into purified powders, extract pastes, solutions for injection, or for oral consumption.

A more detailed description for production of GLS can be found in U.S. Pat. No. 6,316,002, which is hereby incorporated by reference. GLS is also commercially available from Holistol International Ltd. in Hong Kong.

GLS oil is prepared by CO₂ supercritical extraction technology, i.e., the extract is isolated in the CO₂ supercritical state of transformation from gas phase to liquid phase. A more detailed description for production of GLS oil can be found in U.S. Pat. No. 6,440,420, which is hereby incorporated by reference. GLS oil is also commercially available from Holistol International Ltd. in Hong Kong.

The effective amount of GLS oil and/or DHA is a dosage which is useful for ameliorating photoreceptor degeneration. Toxicity and therapeutic efficacy of GLS oil or DHA can be determined by standard pharmaceutical procedures in cell culture or experimental animal models, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. The data obtained from cell culture assays and animal studies can be used in formulating a range of dosages for use in humans.

Generally, appropriate dosages for administering GLS oil may range, for example, from about 0.2 mg/kg body weight/day to about 20 mg/kg body weight/day. In one embodiment, the effective amount of GLS oil is between 1 and 5 mg/kg body weight/day.

GLS oil is preferred to be administered orally. The administration can be in one dose, or at intervals such as three times daily, twice daily, once daily, once every other day, or once weekly. A typical treatment regimen is one administration per day for a period of two or more days, preferably 3-14 days. Dosage schedules for administration of GLS oil can be adjusted based on the individual conditions and needs of the target. Continuous infusions may also be used after the bolus dose. The effects of any particular dosage can be monitored by suitable bioassays.

GLS oil may be administered with another compound that is beneficial to photoreceptor cells, such as DHA. The effective amount of DHA is a dosage which is useful for preventing or ameliorating photoreceptor degeneration. Appropriate dosages for DHA may range, for example, from about 0.2 mg/kg body weight/day to about 20 mg/kg body weight/day. In one embodiment, the effective amount of DHA is between 1 and 5 mg/kg body weight/day.

GLS oil can also be formulated into a pharmaceutical composition with a pharmaceutically acceptable carrier. As used herein the language “pharmaceutically acceptable carrier” is intended to include any and all solvents, solubilizers, fillers, stabilizers, binders, absorbents, bases, buffering agents, controlled release vehicles, diluents, emulsifying agents, humectants, lubricants, dispersion media, coatings, antibacterial or antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary agents can also be incorporated into the compositions.

The pharmaceutical composition of the present invention is formulated to be compatible with its intended route of administration, e.g., oral or parenteral administration. Oral compositions generally include an inert diluent or an edible carrier. They can be enclosed in gelatin capsules or compressed into tablets. For the purpose of oral therapeutic administration, the active compound can be incorporated with a solid carrier and used in the form of tablets, troches, or capsules. Oral compositions can also be prepared using a fluid carrier. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose; a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

Pharmaceutical compositions of GLS oil that are suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the injectable composition should be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the requited particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, such as sodium chloride, sugars, polyalcohols (e.g., manitol, sorbitol, etc.) in the composition. Prolonged absorption of the injectable compositions can be brought about by including in the composition an agent which delays absorption, for example, aluminum monostearate and gelatin.

Sterile injectable solutions can be prepared by incorporating the GLS oil in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

It is especially advantageous to formulate oral or parenteral compositions in dosage unit form for ease of administration and uniformity of dosage. Dosage unit form as used herein includes physically discrete units suited as unitary dosages for the subject to be treated; each unit contains a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals. The pharmaceutical compositions can be included in a container, pack, or dispenser together with instructions for administration.

A person or animal suffering from photoreceptor cell damage/degeneration can be diagnosed by standard clinical procedures such as ophthalmoscopic examination of retina and electroretinography (ERG).

The following experimental designs and result are illustrative, but not limiting the scope of the present invention. Reasonable variations, such as those occur to reasonable artisan, can be made herein without departing from the scope of the present invention. Also, in describing the invention, specific terminology is employed for the sake of clarity. However, the invention is not intended to be limited to the specific terminology so selected. It is to be understood that each specific element includes all technical equivalents which operate in a similar manner to accomplish a similar purpose.

### EXAMPLES

### Example 1

### Animal Model of Photoreceptor Cell Degeneration

Commonly used animal models for inherited retinitis pigmentosain include rd mice, rds mice and RCS rats (Chader G J, Vision Research, 2002, 4:393-396; Deng X G, et al. Chinese Ophthalmic Research. 2006, 24:30-32; Yu T and Yin Z Q, Chinese Journal of Comparative Medicine. 2005, 15: 120-123); the light—induced retinitis pigmentosa in rats, and mice (Rozanowska M and Sarna T. Photochem Phhotobiol. 2005, 81:1305-30); and retinitis pigmentosa induced by MNU (Yoshizawa K et al. Lab Invest. 1999, 79:1359-1367). The common feature of the above described animal models is that the photoreceptor cells in the retinal outer nuclear layer were injured.

The retinitis pigmentosa animal model created by MNU possesses the advantages of simple preparation, abundant animal resources and low cost. Although the retinitis pigmentosa animal model induced by MNU has been reported domestically and abroad in recent years, animal experiments were usually performed with a single, high dosage of MNU in those studies (see e.g., Petrin D et al. IOVS, 2003, 44:2757-2763; and Yang J N et al. Chinese Journal of Ocular Fundus Diseases. 2004, 20: 33-36). The high dosage of MNU will result in irreversible damage of the photoreceptor cells in the out nuclear layer of the subject animals. The threshold dosage, which is the minimal dose that results in the maximum damage of the retinal outer nuclear layer in animals, has not been reported yet. The objective of this study is to observe the changes of the electroretinogram and pathogenic appearance of the retinal outer nuclear layer in rats treated with various dosages of MNU, and to determine the threshold dose for the rat model.

### Materials and Methods

Animals and grouping: 240 50 day-old Sprague Dawley rats, which were divided randomly into 5 test groups and a normal control group with 40 rats in each group. The test groups were treated with 30 mg/kg, 35 mg/kg, 40 mg/kg, 45 mg/kg and 50 mg/kg of MNU respectively. On day 1, 3, 7 and 10 after the MNU treatment, the rats were anaesthetized by intraperitoneal injection of ketamine and chlorpromazine, the pupils were dilated and visual electrophysiological examinations was performed. The anaesthetized rats were then sacrificed by cervical dislocation. Eyeballs were burned and marked for location, enucleated, fixed in the Methacarn solution (60% formaldehyde, 30% chloroform, 10% glacial acetic acid), dissected and stained with Hematoxylin and Eosin (H&E). The damage of the retinal photoreceptors was examined under an optical microscope.

Electroretinogram: Pupils of rats were dilated with ophthalmic solution of mydrin P, followed by dark adaptation for 2 hours. The rats were then anesthetized by intraperitoneal injection with ketamine (50 mg/kg) and chlorpromazine (25 mg/kg). The pupils were dilated again and then dripped with dicaine.

An Neuropack II electrophysiological instrument was employed to measure the visual electrophysiology. Electrodes were installed on the tail, hypoglottis and eye surface, and sodium hydroxymethyl cellulose was dripped into the eyes. The eyes were stimulated with external light source 4 times, the a-wave latent period, b-wave latent period, a-wave amplitudes and b-wave amplitudes at each time point were recorded respectively.

Pathological examination: After fixation in Methacarn solution for 24 hours, the lens was extirpated by incision at the edge of the cornea, incubated in 50% ethanol for 1 hour, dehydrated, embedded (when embedding, the eyeballs were placed horizontally), and sectioned at a thickness of 4 μm. The sections were dewaxed, stained with haematoxylin for 10 min, re-stained with eosin for 5 sec and sealed with neutral gum. The extent of damage of the retinal photoreceptor cell was examined under optical microscope and photographed.

The rating criteria of the retinal photoreceptor cell degeneration induced by MNU were as followed:

a. The retinal outer nuclear layer is arranged in disorder, and the nucleus is damaged, but the thickness of the outer nuclear layer does not decrease significantly, which is defined as grade I damage, and is given a score of 1.

b. The retinal outer nuclear layer is arranged in disorder, and the nucleus is damaged, but the thickness of the damage layer is less than half of the outer nuclear layer, or the entire outer nuclear layer is damaged, and the damaged region accounts for one sixth of the thickness of the entire outer nuclear layer, which is defined as the grade II damage, and is given a score of 2.

c. The entire retinal outer nuclear layer is damaged, and the damaged region accounts for one fourth of the thickness of the entire outer nuclear layer, which is defined as the grade III damage, and is given a score of 4.

d. The entire retinal outer nuclear layer is damaged, and the damaged region accounts for one third of the thickness of the entire outer nuclear layer, which is defined as grade IV damage, and is given a score of 8.

e. The entire retinal outer nuclear layer is damaged, and the damaged region accounts for half of the thickness of the entire outer nuclear layer, which is defined as grade V damage, and is given a score of 16.

### Results:

### Electroretinogram of Rat Retinal Degeneration Induced by Different MNU Dosages

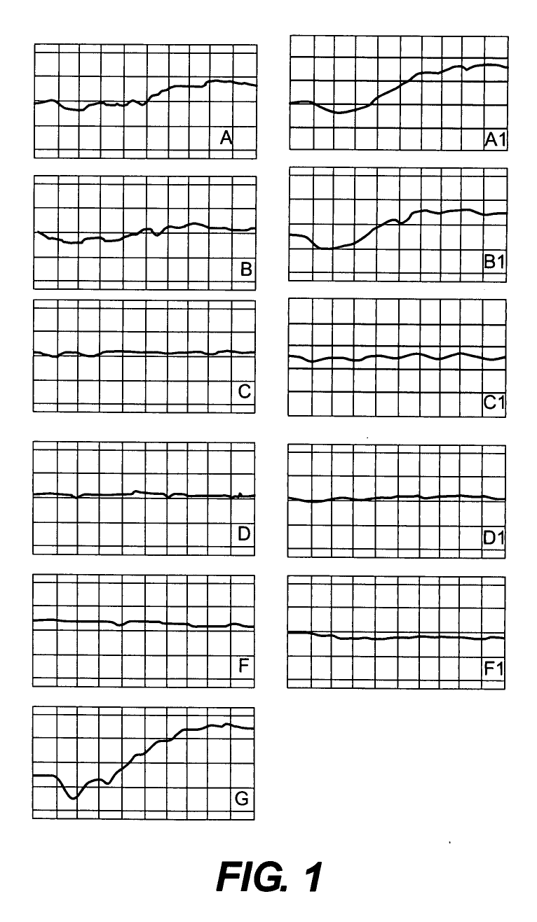

As shown in Table 1 and FIG. 1, at different time points after MNU administration, the a-wave and b-wave latent periods from the normal control group and the test groups treated with 30 mg/kg and 35 mg/kg of MNU, showed no significant difference (P>0.05). The a-wave and b-wave latent periods decreased slightly on day 3 in groups treated with 30 mg/kg and 35 mg/kg of MNU, but the differences are not statistically significant.

However, the a-wave and b-wave latent periods in test groups treated with 40 mg/kg, 45 mg/kg and 50 mg/kg of MNU, are much shorter than those from the normal control group and the test groups treated with 30 mg/kg and 35 mg/kg of MNU (P<0.01). The a-wave latent period and b-wave latent period of the group treated with 40 mg/kg of MNU are not statistically different at the various time points. The a-wave latent period and b-wave latent period of test groups treated with 40 mg/kg and 45 mg/kg of MNU are not statistically different from each other.

Extinguishable visual electrophysiology was presented on day 1, 3 and 7 in the test group treated with 45 mg/kg of MNU, and at all the time points in the test group treated with 50 mg/kg of MNU. The results demonstrated that, at different time points, the MNU dosage of 30 mg/kg and 35 mg/kg had no obvious effect on the a-wave latent period and b-wave latent period of electroretinogram in rats. The MNU dosages of 40 mg/kg, 45 mg/kg and 50 mg/kg obviously shorten the a-wave latent period and b-wave latent period of electroretinogram. The MNU dosage of 40 mg/kg was the lowest dosage which could shorten the a-wave latent period and b-wave latent period of rats electroretinogram to the greatest extent.

TABLE 1
A-Wave and b-Wave Latent Periods of Electroretinogram in Rats Treated with
Different MNU Dosages.

| | | Electrophysiology | | | | |
|---|---|---|---|---|---|---|
| Groups | Latent Period | 12 h | 1 d | 3 d | 7 d | 10 d |
| 30 mg/kg | a-wave | 17.97 ± 2.50 | 17.82 ± 2.59 | 17.44 ± 2.40 | 17.66 ± 2.55 | 17.30 ± 2.24 |
| | b-wave | 73.7 ± 6.94 | 70.41 ± 6.11 | 67.42 ± 7.05 | 70.28 ± 6.02 | 70.31 ± 7.29 |
| 35 mg/kg | a-wave | 17.32 ± 5.87 | 16.98 ± 2.46 | 16.83 ± 2.62 | 17.77 ± 2.25 | 17.46 ± 2.41 |
| | b-wave | 73.61 ± 6.37 | 73.12 ± 6.69 | 68.15 ± 6.68 | 73.37 ± 7.73 | 73.51 ± 5.61 |
| 40 mg/kg | a-wave | 10.53 ± 9.18 | 7.02 ± 9.15 | 3.17 ± 6.69 | 3.37 ± 7.15 | 6.64 ± 8.61 |
| | b-wave | 41.09 ± 35.82 | 27.85 ± 36.29 | 12.20 ± 4.24 | 12.33 ± 26.05 | 27.31 ± 35.56 |
| 45 mg/kg | a-wave | 6.60 ± 9.55 | 0 | 0 | 0 | 6.47 ± 7.33 |
| | b-wave | 25.08 ± 8.55 | 0 | 0 | 0 | 14.42 ± 30.46 |
| 50 mg/kg | a-wave | 0 | 0 | 0 | 0 | 0 |
| | b-wave | | | | | |
| Normal Control | a-wave | 17.92 ± 2.73 | 18.21 ± 2.72 | 17.52 ± 2.15 | 17.81 ± 1.91 | 17.46 ± 5.41 |
| Group | b-wave | 77.91 ± 7.05 | 73.90 ± 7.03 | 73.11 ± 6.46 | 74.86 ± 5.27 | 74.81 ± 5.60 |

As shown in Table 2 and FIG. 1, compared with the normal control group, the a-wave amplitudes and b-wave amplitudes of the rat electroretinogram in the groups treated with 30 mg/kg and 35 mg/kg of MNU decreased at all time points after MNU administration. The decrease was statistically significant on day 1 (P<0.05) and day 3 (P<0.01), but was not statistically significant on 12 h, day 7 and 10 (P>0.05).

In the groups treated with 30 mg/kg and 35 mg/kg of MNU, the a-wave amplitudes and b-wave amplitudes at day 1, 3 and 7 were lower than those at 12 h (P<0.05) and day 10 (P<0.01). The a-wave amplitudes and b-wave amplitudes in the groups treated with 30 mg/kg and 35 mg/kg of MNU, however, were statistically higher than those from the groups treated with 40 mg/kg, 45 mg/kg and 50 mg/kg of MNU at all time points.

The a-wave amplitudes and b-wave amplitudes from the groups treated with 40 mg/kg of MNU were slightly lower at day 3 and 7, but were not statistically different from the other time points. Extinguishable electroretinogram was present at day 1, 3 and 7 d in the group treated with 45 mg/kg of MNU, and at all time points in the group treated with 50 mg/kg of MNU. These results showed that MNU could cause significant decrease in the a-wave amplitudes and b-wave amplitudes of electroretinogram in rats at all tested doses. The dosage of 30 mg/kg and 35 mg/kg MNU had less effect on the a-wave amplitude and b-wave amplitude than the dosages of 40 mg/kg, 45 mg/kg and 50 mg/kg MNU. 40 mg/kg MNU was the initial dosage that could cause maximum decrease of a-wave amplitudes and b-wave amplitudes of electroretinogram. However, there was no significant difference in the a-wave amplitudes and b-wave amplitudes among rats treated with 40 mg/kg, 45 mg/kg or 50 mg/kg MNU.

TABLE 2
A-Wave Amplitudes and b-Wave Amplitudes of Electroretinogram from Rats
Treated with Different MNU Dosages (−x ± s, n = 10)

| | | Electrophysiology | | | | |
|---|---|---|---|---|---|---|
| Groups | Amplitude | 12 h | 1 d | 3 d | 7 d | 10 d |
| 30 mg/kg | a-wave | 42.24 ± 7.71 | 34.10 ± 6.35 | 33.14 ± 9.15 | 41.11 ± 10.14 | 44.17 ± 11.13 |
| | b-wave | 195.8 ± 33.45 | 153.6 ± 28.76 | 132.0 ± 24.85 | 156.5 ± 34.85 | 188.7 ± 27.7 |
| 35 mg/kg | a-wave | 40.34 ± 8.37 | 33.20 ± 12.11 | 30.38 ± 9.61 | 36.75 ± 7.28 | 40.76 ± 8.92 |
| | b-wave | 185.3 ± 23.41 | 107.52 ± 17.38 | 104.88 ± 21.83 | 135.44 ± 22.58 | 168.7 ± 32.14 |
| 40 mg/kg | a-wave | 17.18 ± 15.90 | 9.44 ± 12.38 | 3.62 ± 7.66 | 4.23 ± 9.27 | 9.54 ± 12.89 |
| | b-wave | 54.40 ± 46.33 | 23.09 ± 30.15 | 12.23 ± 26.02 | 13.14 ± 27.85 | 24.25 ± 31.58 |
| 45 mg/kg | a-wave | 0 | 0 | 0 | 0 | 4.89 ± 10.48 |
| | b-wave | 0 | 0 | 0 | 0 | 11.31 ± 23.93 |
| 50 mg/kg | a-wave | 0 | 0 | 0 | 0 | 0 |
| | b-wave | | | | | |
| Normal Control | a-wave | 45.05 ± 7.33 | 46.95 ± 6.72 | 45.46 ± 9.00 | 43.71 ± 7.61 | 44.94 ± 7.66 |
| Group | b-wave | 23.05 ± 47.40 | 234.6 ± 44.50 | 242.3 ± 37.92 | 246.8 ± 40.22 | 238.1 ± 41.80 |

A shown in Table 3, with an increase of the MNU dosage from 40 mg/kg to 50 mg/kg, the number of eyes with extinguished electroretinogram, which was caused by retinal photoreceptor damage induced by MNU, increased at different time points. While rats treated with 30 mg/kg and 35 mg/kg of MNU did not exhibit any extinguished electroretinogram. The results demonstrated that the dosage of 50 mg/kg MNU would cause the maximum rat retinal photoreceptor damage, while the dosages of 30 mg/kg and 35 mg/kg MNU cause less damages to the rat retinal photoreceptor, and the dosage of 40 mg/kg MNU was the initial dosage which could cause maximum damage of rat retinal photoreceptor.

TABLE 3
Extinguished Electroretinogram in Rats Treated with Different MNU
Dosages (−x ± s, n = 10)

| | Number of Eyes with extinguished | | | | |
|---|---|---|---|---|---|
| | electroretinogram. | | | | |
| Groups | 12 h | 1 d | 3 d | 7 d | 10 d |
| 30 mg/kg | 0 | 0 | 0 | 0 | 0 |
| 35 mg/kg | 0 | 0 | 0 | 0 | 0 |
| 40 mg/kg | 4 | 6 | 8 | 8 | 6 |
| 45 mg/kg | 6 | 10 | 10 | 10 | 8 |
| 50 mg/kg | 10 | 10 | 10 | 10 | 10 |
| Normal | 0 | 0 | 0 | 0 | 0 |
| Control | | | | | |
| Group | | | | | |

Pathology of Retinal Photoreceptor Cells in Rat Treated with Different Dosages of MNU

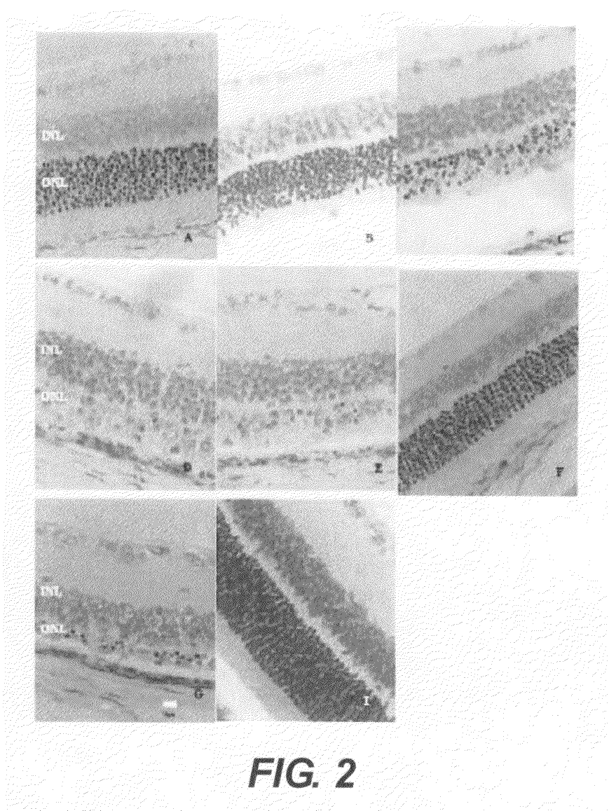

As shown in Table 4 and FIG. 2, the MNU-induced damage to rat retinal photoreceptor cells occurred in the form of apoptosis. The apoptosis occurred first in the outer nuclear layer. A fraction of outer nuclear layer cells were damaged and died, resulting in a mis-arrangement of the outer nuclear layer. Next, the photoreceptor cells in the entire outer nuclear layer died around the posterior pole The apoptosis then moved outward, to the central and peripheral retina.

The damage induced by high doses of MNU first occurred at the posterior pole, around the optic nerve papilla, then moved outward to the central retina. No recovery of outer nuclear layer cells was observed at day 7 to day 10.

The dosages of 30 mg/kg and 35 mg/kg MNU led to relatively small damage to the rat retinal photoreceptor cell at different time points, which only resulted in apoptosis and disorder of the outer nuclear layer. The decrease in the number of outer nuclear layers generally was not more than half of the entire outer nuclear layer. Recovery of the outer nuclear layer occurred on day 7 and day 10. The number of dead cells decreased significantly, and the outer nuclear layer lined up in order. The dosage of 35 mg/kg MNU caused damage of retinal photoreceptor cell on day 3. The damage on day 3 was more severe than that on day 1 and day 10 in the same group, and was more severe than that on day 1, 7 and 10 d in the 30 mg/kg MNU group.

The damage caused by dosages of 40 mg/kg, 45 mg/kg and 50 mg/kg MNU was more severe than that caused by the lower doses. The high doses led to damages in the entire outer nuclear layer, which occurred first on the posterior pole, around the optic nerve papilla, then developed outward to the central and peripheral retina. No obvious repair on outer nuclear layer was observed on day 7 or day 10. The damage caused by the dosage of 40 mg/kg, 45 mg/kg and 50 mg/kg MNU at different time points were clearly more severe than those in the 30 mg/kg (P<0.05) and 35 mg/kg MNU (P<0.01) dosage group. The damage in animals treated with 45 mg/kg and 50 mg/kg MNU is more severe than that in animals treated with 40 mg/kg of MNU at 12 h and day 1. The damage in animals treated with 50 mg/kg MNU is more severe than that in animals treated with 40 mg/kg of MNU on day 3. There were no significant difference on day 7 and day 10 in animals treated with 40 mg/kg, 45 mg/kg, or 50 mg/kg MNU (P>0.06). The extent of damage to rat retinal photoreceptor cells was 50 mg/kg >45 mg/kg >40 mg/kg.

These results demonstrated that MNU-induced damage to rat retinal photoreceptor cells aggravated over time. The damage caused by 30 mg/kg or 35 mg/kg MNU was relatively light, while the damage caused by 40 mg/kg, 45 mg/kg and 50 mg/kg MNU was much more severe. The most severe damage was caused by the dosages of 45 mg/kg and 50 mg/kg MNU. Among the various time points, the damages on day 3, 7 and 10 were more severe than the damages at 12 h and day 1. The minimum dosage that could cause damage to the entire outer nuclear layer cell of rat retinal was 40 mg/kg.

TABLE 4
Pathological Evaluation of Damages to Rat Retinal Photoreceptor Cells Induced
by Different MNU Dosages (−x ± s, n = 10)

| | Damage score | | | | |
|---|---|---|---|---|---|
| Groups | 12 h | 1 d | 3 d | 7 d | 10 d |
| 30 mg/kg | 0.20 ± 0.42 | 0.40 ± 0.52 | 0.40 ± 0.52 | 0.20 ± 0.42 | 0 |
| 35 mg/kg | 0.20 ± 0.42 | 0.60 ± 0.52 | 0.80 ± 0.42 | 0.40 ± 0.52 | 0 |
| 40 mg/kg | 1.00 ± 0.67 | 2.20 ± 1.03 | 4.60 ± 3.10 | 7.60 ± 6.29 | 7.40 ± 6.44 |
| 45 mg/kg | 1.6 ± 0.52 | 4.00 ± 2.31 | 6.80 ± 5.27 | 9.60 ± 5.95 | 9.20 ± 6.20 |
| 50 mg/kg | 2.40 ± 1.43 | 6.80 ± 5.27 | 10.04 ± 5.06 | 11.20 ± 5.27 | 11.20 ± 5.27 |
| Normal | 0 | 0 | 0 | 0 | 0 |
| Control | | | | | |
| Group | | | | | |

### Discussion

MNU is a nitroso compound that exists ubiquitously in the environment. MNU has a strong carcinogenic, teratogenic and mutagenic tendency, and is mainly used to induce breast carcinoma and reproductive organ tumor in animals for tumor research. Herrold et al. first found that MNU could induce rat retina degeneration, and it has been proven that death and damage of retinal photoreceptor cell could occur even with treatment with MNU intraperitoneal injection with a single dose, the mechanism of which was to restrict the nucleus DNA inward turning resulting in the final apoptosis of the photoreceptor cell, accompanied by the downward adjustment of Bcl-2, upward adjustment of Bax and the activation of Caspase-3, -6, -8 pathway (Yoshizawa K and Tsubura A., Nippon Ganka Gakkai Zasshi. 2005, 109: 327-37). Therefore, this model is an ideal animal model for retinal degeneration research.

During 2003, it was reported by Kiuchi et al. (Kiuchi K et al. Current Eye Research, 2003, 26: 355-362). that the niacinamide could suppress the SD animal model with retinal photoreceptor cell apoptosis induced by 60 mg/kg of MNU. In 2004, Moriguchi et al. (Moriguchi K et al. Ophthalmic Res. 2004, 26: 98-105) reported this kind of SD animal model induced by 50 mg/kg of MNU could be suppressed by diet supplement with DHA. However, in the above mentioned articles, only a single dosage of MNU was used, and the authors did not answer questions as to why this dosage was adopted in their studies and what was the advantage of the dosage.

The present results demonstrated that different dosages of MNU could cause decreases in a-wave amplitudes and b-wave amplitudes of electroretinogram. The dosage of 30 mg/kg and 35 mg/kg MNU affected the a-wave amplitudes and b-wave amplitudes to a lesser extent than the dosages of 40 mg/kg, 45 mg/kg and 50 mg/kg. The minimal MNU dosage that could cause the maximum decrease of the a-wave amplitudes and b-wave amplitudes is 40 mg/kg. Additionally, the pathologic observation is in agreement with the electrophysiological results. At all time points, the pathological damages to the rat retinal photoreceptor cells induced by 30 mg/kg and 35 mg/kg of MNU were slight; and the pathological damages induced by 40 mg/kg, 45 mg/kg and 50 mg/kg of MNU were much more severe, with the most severe damages caused by 45 mg/kg and 50 mg/kg of MNU. The electrophysiological and pathologic results demonstrated that the minimum dosage which could cause most severe damage to the retinal outer nuclear layer of rats was 40 mg/kg of MNU. Therefore, the optimal dosage of MNU for creating a SD rat model of retinal photoreceptor cell damage is about 40 mg/kg. The animal model can be used for investigating the inhibitory effect and mechanism of certain drugs on the damage of the retinal outer nuclear layer photoreceptor cells.

### Example 2

### Effect of the Ganoderm Lucidum Spores Oil on the Electroretinogram and Pathologic Appearance of Photoreceptor Damage Induced by MNU in Rats

### Materials and Methods:

Animals and grouping: One hundred and five 50-day-old Sprague Dawley rats were used in the experiment. Twenty-five rats were selected randomly as the normal control (receiving an excipient via gastric gavage). Eighty rats were used for establishing the photoreceptor cell degeneration model and were treated by intraperitoneal injection with a single dose of MNU at 40 mg/kg. The MNU-injected rats were randomly divided into four experimental groups:

(1) GLS oil treatment group (receiving GSL oil and an excipient via gastric gavage);

(2) GLS oil+DHA treatment group (receiving GSL oil, DHA, and an excipient via gastric gavage);

(3) DHA control group (receiving DHA and an excipient via gastric gavage;

(4) Model control group (receiving an excipient via gastric gavage).

The excipient used in this experiment is hydroxypropyl methyl cellulose. The treatment started three days before the administration of MNU (day-3). Each animal in Groups 1-3 received a daily gastric feeding of GSL oil only, GSL oil and DHA (2 mg/kg), or DHA (2 mg/kg) only, in 1 ml 0.5% hydroxypropyl methyl cellulose. Animals in Group 4 received 1 ml 0.5% hydroxypropyl methyl cellulose. Animals in the normal control group did not receive anything.

The animals were anesthetized by intraperitoneal injection with ketamine and chlorpromazine at 24 h, 3 d, 7 d, and 10 d after the MNC injection. The pupils were dilated and a visual electrophysiological examinations was performed. The rats were then sacrificed by cervical dislocation. Eyeballs were burned for identification, removed, fixed in the Methacarn solution (60% formaldehyde, 30% chloroform, 10% glacial acetic acid), dissected, and stained with Hematoxylin & Eosin (HE).

Electroretinogram examination: The pupils of rats were dilated as described in Example 1. The electroretinogram examinations were conducted under the following conditions:

| | |
|---|---|
| SENS 1CH | 200 uv/DIV |
| HIGH CUT | 200 Hz |
| LOW CUT | 0.5 Hz |
| HUM FILTER | OFF |
| SWEET TIME | 10 ms/DIV |
| Mon SPEED | 10 ms/DIV |
| AVER COUNT MEM | 0 |
| AUTO START | OFF |
| STIM RATE | EXT |
| RANDOM | OFF |
| SWEET DELAY | 0% |
| VISUSAL STIM | EXT_FLASH |

A Neuropack II electrophysiological analyzer was employed to measure the visual electrophysiology. Electrodes were installed on the tail, hypoglottis and eye surface, and sodium hydroxymethyl cellulose was dripped, the eyes were stimulated with external light source 4 times, the a-wave latent period, b-wave latent period, a-wave amplitude and b-wave amplitude at each time point were recorded.

Pathological examinations: The pathological examinations were carried out as described in Example 1.

### Results

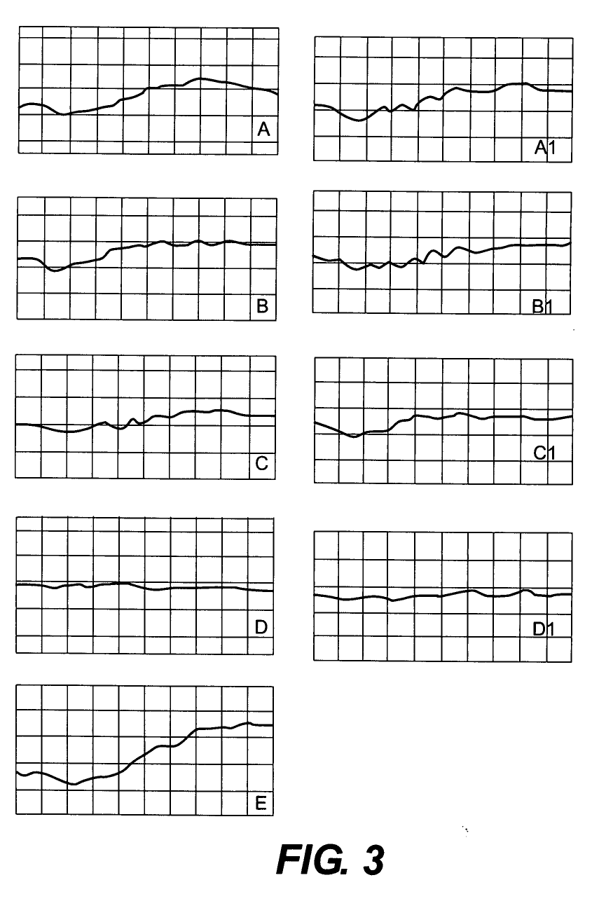

Electroretinogram: As shown in Table 5 and FIG. 3, except for the GLS oil treatment group on day 1 and day 10, the a-wave latent period and b-wave latent period shortened significantly in the MNU-treated groups at various time points compared with the normal control group, with P<0.05 or P<0.01. The shortening of the a-wave latent period and b-wave latent was more apparent in all the treated groups on day 3 and day 5. There was no significant difference between the a-wave latent period and b-wave latent period among different time points from the same group. Except for the b-wave amplitudes on day 1 and day 10 in the GLS oil treatment group, the a-wave amplitudes and b-wave amplitudes decreased significantly in the MNU-treated groups at each time point, compared with the normal control group.

The b-wave amplitudes in the treatment groups were apparently higher than that in the model control group at each time points (P<0.05 or P<0.01). The a-wave amplitudes in the GLS oil treatment group were apparently higher than those in the model control group (P<0.05), but were not significantly different from those in the GLS oil/DHA group and DHA group (P>0.05). The decrease of the a-wave amplitudes and b-wave amplitudes was more evident at day 3 and day 5 in all MNU-treated groups. The extent of the effect was GLS oil treatment group>the DHA treatment group>GLS oil/DHA treatment group>the model control group.

These results demonstrated that different treatments did not have much effect on the a-wave latent period and b-wave latent period of the MNU-treated rat retinal outer nuclear layer photoreceptor cells. GLS oil and DHA suppressed, at a different degree, the MNU-induced reduction of a-wave amplitudes and b-wave amplitudes of electroretinogram of rat retinal outer nuclear layer photoreceptor cells.

TABLE 5
The Effects of Different Compounds on the Electroretinogram of Rat Retinal
Degeneration Induced by MNU (±s, n = 10)

| | Time | a-Wave Latent | b-Wave | a-Wave | b-Wave |
|---|---|---|---|---|---|
| Groups | (day) | Period | Latent Period | Amplitude | Amplitude |
| GLS oil treatment | 1 | 14.16 ± 7.65 | 57.63 ± 31.49 | 28.08 ± 18.68 | 94.91 ± 69.19 |
| group | 3 | 10.46 ± 9.20 | 43.28 ± 37.61 | 2.23 ± 19.92 | 76.10 ± 67.61 |
| | 5 | 10.35 ± 9.13 | 42.92 ± 37.46 | 16.83 ± 14.96 | 67.78 ± 55.18 |
| | 7 | 10.45 ± 9.23 | 43.36 ± 38.81 | 19.70 ± 22.94 | 72.62 ± 65.12 |
| | 10 | 12.43 ± 8.69 | 51.52 ± 36.15 | 22.16 ± 18.16 | 86.95 ± 63.53 |
| DHA treatment group | 1 | 10.35 ± 9.02 | 43.92 ± 38.08 | 18.43 ± 17.94 | 56.90 ± 61.16 |
| | 3 | 8.64 ± 9.91 | 35.10 ± 37.34 | 14.13 ± 15.71 | 59.43 ± 65.22 |
| | 5 | 8.30 ± 8.81 | 34.92 ± 36.97 | 10.27 ± 11.15 | 47.85 ± 52.05 |
| | 7 | 9.01 ± 9.56 | 36.24 ± 38.45 | 16.61 ± 18.95 | 53.93 ± 58.06 |
| | 10 | 10.42 ± 9.10 | 43.23 ± 37.88 | 19.70 ± 17.97 | 64.71 ± 57.60 |
| GLS oil + DHA | 1 | 10.35 ± 9.05 | 43.59 ± 37.90 | 16.79 ± 15.67 | 63.99 ± 57.56 |
| treatment group | 3 | 8.37 ± 8.93 | 35.33 ± 37.72 | 12.80 ± 14.31 | 48.29 ± 51.77 |
| | 5 | 6.47 ± 8.98 | 34.94 ± 38.12 | 9.11 ± 10.02 | 44.73 ± 47.75 |
| | 7 | 8.52 ± 9.15 | 36.58 ± 38.75 | 12.06 ± 13.26 | 49.87 ± 53.02 |
| | 10 | 10.22 ± 8.88 | 41.92 ± 36.39 | 16.43 ± 14.77 | 61.47 ± 53.49 |
| Model control group | 1 | 6.21 ± 10.17 | 28.7 ± 37.64 | 9.41 ± 12.34 | 21.43 ± 27.97 |
| | 3 | 3.33 ± 7.03 | 13.59 ± 28.67 | 3.71 ± 7.84 | 10.09 ± 21.62 |
| | 5 | 3.02 ± 6.37 | 14.04 ± 29.84 | 3.49 ± 7.38 | 9.90 ± 21.00 |
| | 7 | 3.62 ± 7.64 | 14.44 ± 30.47 | 4.23 ± 9.00 | 11.17 ± 23.55 |
| | 10 | 6.76 ± 11.20 | 27.77 ± 36.17 | 9.34 ± 12.38 | 20.98 ± 27.31 |
| Normal control group | 1 | 17.23 ± 2.69 | 75.16 ± 5.84 | 45.87 ± 7.97 | 243.0 ± 42.97 |
| | 3 | 17.16 ± 2.30 | 75.72 ± 6.04 | 46.42 ± 8.92 | 240.5 ± 40.55 |
| | 5 | 17.38 ± 2.67 | 74.76 ± 6.68 | 45.40 ± 5.98 | 235.6 ± 39.71 |
| | 7 | 17.26 ± 1.91 | 73.96 ± 7.20 | 45.28 ± 7.75 | 240.3 ± 41.07 |
| | 10 | 17.10 ± 2.44 | 73.85 ± 6.23 | 44.91 ± 7.35 | 242.8 ± 45.69 |

As shown in Table 6, among different time points, the GLS oil treatment group had the smallest number of eyes with extinguishable electroretinogram. The DHA-treatment group had about the same number of eyes with extinguishable electroretinogram as the GLS oil/DHA treatment group. The model control group had the largest number of eyes with extinguishable electroretinogram.

TABLE 6
Extinguished Electroretinogram Induced by MNU in Different Compound
Therapeutic Groups (−x ± s, n = 10)

| | Number of Eyes with | | | | |
|---|---|---|---|---|---|
| | Extinguished Electroretinogram | | | | |
| Groups | day 11 | day 3 | day 5 | day 7 | day 10 |
| GLS oil treatment | 2 | 4 | 4 | 4 | 3 |
| group | | | | | |
| DHA treatment | 4 | 5 | 5 | 5 | 4 |
| group | | | | | |
| GLS oil + DHA | 4 | 5 | 5 | 5 | 4 |
| treatment group | | | | | |
| Model control | 6 | 8 | 8 | 8 | 6 |
| group | | | | | |
| Normal control | 0 | 0 | 0 | 0 | 0 |
| group | | | | | |

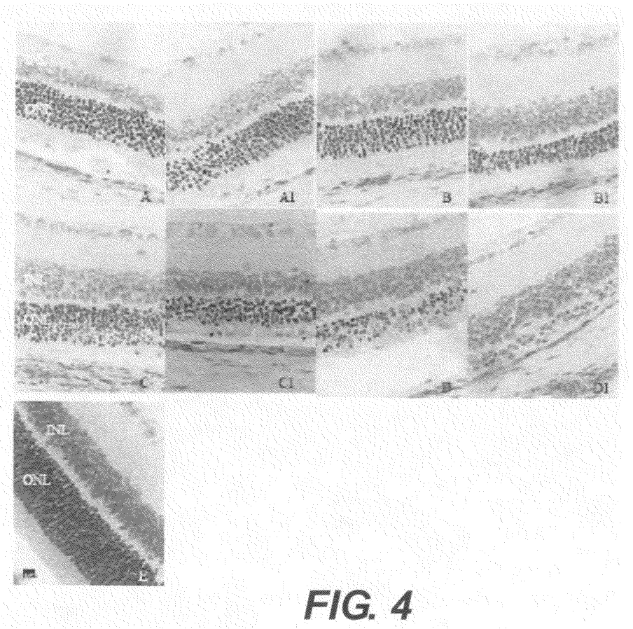

Pathologic examination: As shown in Table 7 and FIG. 4, compared to the model control group, the MNU-induced rat retinal photoreceptor cells damage was alleviated significantly in all treatment groups (P<0.05). This result demonstrated that both GLS oil and DHA could suppress the MNU-induced pathologic damage of rat retinal outer nuclear layer photoreceptors.

TABLE 7
The Effects of GLS Oil on MNU-Induced Retinal Photoreceptor Cell Pathological
Damage (−x ± s, n = 10).

| | The Damage Degree of the Retinal | | | | |
|---|---|---|---|---|---|
| | Photoreceptor Cell Pathology | | | | |
| Groups | day 1 | day 3 | day 5 | day 7 | day 10 |
| GLS oil | 1.00 ± 0.67 | 1.80 ± 1.32 | 2.60 ± 2.37 | 2.40 ± 2.55 | 2.2 ± 2.70 |
| treatment group | | | | | |
| DHA treatment | 1.40 ± 0.52 | 2.2 ± 1.32 | 2.9 ± 2.85 | 2.80 ± 2.97 | 2.60 ± 2.95 |
| group | | | | | |
| GLS oil + DHA | 1.40 ± 0.52 | 2.3 ± 1.34 | 3.00 ± 2.26 | 2.80 ± 2.48 | 2.70 ± 2.54 |
| treatment group | | | | | |
| Model control | 2.2 ± 1.03 | 4.60 ± 3.10 | 7.80 ± 6.12 | 7.60 ± 6.29 | 7.40 ± 6.45 |
| group | | | | | |
| Normal control | 0 | 0 | 0 | 0 | 0 |
| group | | | | | |

### Discussion

MNU causes retinal outer nuclear layer damage by specifically targeting the retinal photoreceptor cell and restricting the inward rotation of nuclear DNA, finally resulting in the apoptosis of the photoreceptor cell. MNU also leads to the up-regulation of Bax, the down-regulation of Bcl-2, and the activation of Caspase-3, Caspas-6, and Caspas-8 (Yoshizawa K et al., Lab Invest. 1999, 79:1359-1367). It was found in the preliminary experiment that 40 mg/kg was the lowest MNU dose that could induce maximum damage of the rat retinal photoreceptor cell, and that initial damage of the photoreceptor cell occurred 1 day after MNU administration, while the most severe damage occurred between day 3 to day 5.

The electroretinogram reflects the function of retinal photoreceptor cell (rod cell and cone cell). The a-wave mainly indicates the function of rod cell, while the b-wave shows the function of cone cell.

Results in Example 2 showed that the b-wave amplitudes were significantly increased at different time points in the GLS oil treatment group, DHA treatment group, and GLS oil/DHA treatment group. The a-wave amplitudes were increased significantly at different time points in the GLS oil-treatment group (P<0.05 or P<0.01). In addition, the damage of rat retinal outer nuclear layer induced by MNU was alleviated evidently at different time points from the groups treated with compounds, with P<0.05. These results demonstrated that Ganoderm lucidum spores oil and DHA could alleviate the damage of rat retinal photoreceptor cell induced by MNU to various extents, and facilitate the function recovery of the photoreceptor cell. The effectiveness of the photoreceptor cells protection was in the following sequence: Ganoderm lucidum spores oil treated group>DHA treated group>Ganoderm lucidum spores oil plus DHA treat group>model control group.

The disadvantages of supplementing DHA in diet are (1) it is difficult to accurately determine the intake amount, and (2) DHA is susceptible to oxidation. These disadvantages can be overcome by supplementing DHA through via gastric gavage.

Supercritical fluid extraction/gas chromatography/mess spectrography (SFE—GC—MS) analysis showed that Ganoderm lucidum spores oil contains various unsaturated fatty acids (Tian G et al., China Oils and Fats, 2003, 28: 44-45). The unsaturated fatty acid accounts for 68.42% of the total amount, in which linoleic acid and oleic acid accounts for 18.82% and 43.63%, respectively. While the saturated fatty acid is mainly composed of palmitic acid and stearic acid.

In 2003, Semenova et al. (Semenova E M and Converse C A. Vision Research. 2003, 43:3063-3067) reported that retinal photoreceptor matrix is rich in oleic acid, and the binding ability of oleic acid to IRBP is stronger than DHA. The function of interphotoreceptor retinoid—binding protein (IRBP) is to bind the 11-cis-retinene and all—trans-retinene, and to facilitate their transportation between photoreceptor cell and retinal pigment epithelium. It was reported (Chen Y et al., J. Biol. Chemistry 1996, 271:20507-20515) that by binding to IRBP, DHA releases the 11-cis-retinene upon IRBP migrating to the photoreceptor cell, DHA can stabilize the binding of IRBP with the photons. The 11-cis-retinene binds to opsin to form rhodopsin, the latter plays an important role in the visual forming. To date, the biology function of the binding of oleic acid to IRBP remains unclear. The oleic acid is a monounsaturated fatty acid, which has the effects of decreasing blood lipids, lowing blood sugar levels, reducing cholesterol, preventing coronary heart disease, and maintaining nerve cell integrity and so on (Zhang W M et al., Journal of Cereals and Oils 2005, 3:13-15). The oleic acid possibly plays important roles in the photon delivery, disc membrane formation and disc membrane integrity maintenance.

Ruiyi Chen et al. (Chen R Y et al. Journal of Chinese Modern Medicine 2005, 2: 304-305) reported that Ganoderm lucidum spores oil can prevent the liver damage induced by CCL4 in 2005. The anti free radical and anti-oxidative effect of Ganoderm lucidum spores oil have an important role in preventing the rat retinal photoreceptor cell damage induced by MNU.

The embodiments illustrated and discussed in this specification are intended only to teach those skilled in the art the best way known to the inventors to make and use the invention. Nothing in this specification should be considered as limiting the scope of the present invention. The above-described embodiments of the invention may be modified or varied, and elements added or omitted, without departing from the invention, as appreciated by those skilled in the art in light of the above teachings. It is therefore to be understood that, within the scope of the claims and their equivalents, the invention may be practiced otherwise than as specifically described.

What is claimed is:

1. A method for ameliorating photoreceptor cell degeneration in a mammal comprising:
administering to said mammal a composition comprising an effective amount of germination activated sporoderm-broken Ganoderma lucidum spores oil (GLS oil);
wherein said mammal has photoreceptor cell degeneration.

2. The method according to claim 1, wherein said mammal is a human.

3. The method according to claim 1, wherein said mammal is a rodent.

4. The method according to claim 1, wherein said photoreceptor cell degeneration is caused by retinitis pigmentosa.

5. The method according to claim 1, wherein said photoreceptor cell degeneration is caused by N-methyl-N-nitrosourea induction.

6. The method according to claim 1, wherein said effective amount of GLS oil is between 0.2 and 20 mg/kg body weight/day.

7. The method according to claim 6, wherein said effective amount of GLS oil is between 1 and 5 mg/kg body weight/day.

8. The method according to claim 1, wherein said composition further comprises an effective amount of docosahexaenoic acid (DHA).

9. The method of claim 8, wherein said effective amount of DHA is between 0.2 and 20 mg/kg body weight/day.

10. The method according to claim 9, wherein said effective amount of DHA is between 1 and 5 mg/kg body weight/day.

11. The method according to claim 1, wherein said composition is administered orally.

12. A method for treating retinitis pigmentosa (RP) in a mammal comprising:
administering to a mammal suffering from RP a composition comprising an effective amount of germination activated sporoderm-broken Ganoderma lucidum spores oil (GLS oil).

13. The method according to claim 12, wherein said mammal is a human.

14. The method according to claim 13, wherein said effective amount of GLS oil is between 0.2 and 20 mg/kg body weight/day.

15. The method according to claim 14, wherein said effective amount of GLS oil is between 1 and 5 mg/kg body weight/day.

16. The method according to claim 12, wherein said composition further comprises an effective amount of docosahexaenoic acid (DHA).

17. The method of claim 16, wherein said effective amount of DHA is between 0.2 and 20 mg/kg body weight/day.

18. The method according to claim 17, wherein said effective amount of DHA is between 1 and 5 mg/kg body weight/day.

19. The method according to claim 12, wherein said composition is administered orally.

20. The method according to claim 12, wherein said RP is induced by N-methyl-N-nitrosourea.
